(19) United States
(12) Patent Application Publication
(10) Pub. No.: US 2025/0367233 A1
(43) Pub. Date: Dec. 4, 2025

(54) NOVEL PHARMACEUTICAL COMPOSITION CONTAINING IRON AND MEGLUMINE

(71) Applicant: Christopher Newton Jobdevairakkam, Monroe Twp, NJ (US)

(72) Inventor: Christopher Newton Jobdevairakkam, Monroe Twp, NJ (US)

(21) Appl. No.: 19/052,252

(22) Filed: Feb. 12, 2025

Related U.S. Application Data
(60) Provisional application No. 63/555,783, filed on Feb. 20, 2024.

(51) Int. Cl. A61K 33/26; A61K 9/20; A61K 9/48; A61K 31/133

(52) U.S. Cl. CPC A61K 33/26; A61K 9/2013; A61K 9/4858; A61K 31/133

(57) ABSTRACT

The present invention is related to a novel pharmaceutical product containing iron and meglumine. In certain embodiments, the present invention is related to a novel injectable dosage form comprising ferric meglumine as a parenteral iron supplement to treat iron deficiency anemia. In certain embodiments, the present invention is related to a novel oral dosage form comprising ferric meglumine as iron supplement and/or phosphate binder.

### CROSS REFERENCE TO RELATED APPLICATIONS

The present application claims the benefit of U.S. Provisional Application No. Ser. No. 63/555,783 filed on Feb. 20, 2024, the entire disclosure of which is herein incorporated by reference.

### FIELD OF INVENTION

In certain embodiments, the present invention relates to a novel iron product, ferric Meglumine, pharmaceutical compositions thereof and processes for preparing the same.

### BACKGROUND OF THE INVENTION

### A. Pharmaceutical Iron Products

Iron is one of the most essential elements found in biological organisms. Iron containing pharmaceutical products are known for use as iron supplements as well as phosphate binders. Iron supplements are necessary to replenish the blood iron or iron stores in patients having ailments such as iron deficiency anemia (IDA), chronic kidney disease (CKD), and patients undergoing dialysis for any other condition. Iron can be supplemented through oral or parenteral route of administration. Iron containing pharmaceutical products are also known to be used as phosphate binders to reduce intake of dietary phosphates.

### B. Parenteral Iron Products

Presently available parenteral iron products include iron dextran (INFED®), iron sucrose complex in sucrose (VENOFER®), sodium ferric gluconate complex in sucrose, (FERRLECIT®), ferumoxytol (FERRAHEME®), ferric derisomaltose (MONOFERRIC®), ferric carboxymaltose (INJECTAFER® are used to supplement iron through parenteral administration.

### C. Orally Administered Iron Products

Orally administered iron products are predominantly used as iron supplements, or phosphate binders. Ferric Citrate (AURYXIA®), and sucroferric oxyhydroxide (VELPHORO®) are used as phosphate binders; ferric hexacyanoferrate (II) aka prussian blue (RADIOGARDASE®) is used as a decontamination agent to eliminate thallium or radioactive cesium. Ferric maltitol (ACCRUFER®), and other over-the-counter drugs such as ferrous sulphate, ferrous fumarate, ferrous ascorbate, ferrous gluconate, ferrous asparto glycinate, carbonyl iron are useful as supplements to treat iron deficiency anemia.

Each iron containing pharmaceutical product has its own merits and demerits. Iron dextran has a history of incidents, and currently INFED® is marketed with boxed warning to alert patients about severe allergic reaction. Such rare allergic reaction was ascertained to the quality of dextran used in the manufacture of Iron dextran product. Other injectable iron products pose a risk of hypersensitivity reactions such as anaphylaxis. As reported in JAMA. 2015; 314(19):2062-2068. doi:10.1001/jama.2015.15572, the risk for anaphylaxis at first exposure was 68 per 100,000 persons for iron dextran (95% CI, 57.8-78.7 per 100,000) and 24 per 100,000 persons for all non-dextran IV iron products combined (iron sucrose, gluconate, and ferumoxytol) (95% CI, 20.0-29.5 per 100,000), with an adjusted odds ratio (OR) of 2.6 (95% CI, 2.0-3.3; P<0.001). Venofer® and Ferrlecit® contains large amount of sucrose in the drug product, administering such high doses of sucrose is not a favorable treatment option available for diabetic patients' requiring supplemental iron.

Meglumine is a base used in salt formation of drugs used in oral and injectable dosage forms. Some of the approved drugs that contain meglumine are delafloxacin meglumine (BAXDELA®), diatrizoate meglumine (CYSTOGRAPFIN®), fosapprepitant dimeglumine (EMEND®) & (FOCINVEX®), gadobenate dimeglumine (MULTIHANCE®), gadoterate meglumine (DOTAREM®) & (CLARISCAN®), lothalamate meglumine (CONRAY®), tafamidis meglumine (VYNDAQEL®), flunixin meglumine (VETAMEG®). Structure of Meglumine:

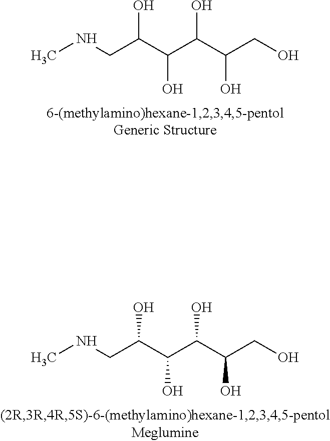

Other Names of Meglumine: N-Methyl-D-glucamine; N-Methylglucamine; Methylglucamine; Meglumin; D-Glucitol, 1-deoxy-1-(methylamino)-; N-Methyl-D-(−) glucamine; d(−) N-Methylglucamine; Sorbitol, 1-deoxy-1-methylamino-.

Developing a safer parenteral iron supplement that has low or no sugar residue is always preferable. This invention presents a new Iron product containing ferric hydroxide and meglumine.

The term ferric hydroxide expressed herein can be defined as an iron species that includes various forms of ferric hydroxide, including, ferric hydroxide, polymeric ferric hydroxide, ferric oxyhydroxide, polymeric ferric oxyhydroxide, ferric hydroxide gel, hydrated ferric oxide, partially neutralized ferric salts and partially neutralized polymeric ferric salts. Optionally the ferric hydroxide may contain ferrous ions at a level below 30% to total Iron, preferably 10% to total Iron, most preferably below 1% to total Iron.

### SUMMARY OF INVENTION

In certain embodiments, the present invention relates to a compound that is a combination of iron and meglumine. In certain aspects, the compound comprises ferric iron and meglumine.

In an aspect, the compound as described above contains one or more components selected from oxide, hydroxide, water, sodium, chloride, solvent, organic impurities, elemental impurities.

In an aspect, the compound as described above is substantially free from organic impurities.

In an aspect, the compound as described above is substantially free from elemental impurities or having elemental impurities at a level below the acceptance level for parenteral products.

In an aspect of the compound as described above, the iron is ferric iron. In an aspect of the compound as described above, the iron is a mixture of ferric and ferrous iron. In an aspect of the compound as described above, the iron is present in the form of iron hydroxide. In an aspect of the compound as described above, the iron hydroxide is a ferric iron compound and/or combination of ferric iron and ferrous iron thereof. In an aspect of the compound as described above, the iron is one or more selected from the group consisting of ferric hydroxide, polymeric ferric hydroxide, ferric oxyhydroxide, polymeric ferric oxyhydroxide, ferric hydroxide gel, hydrated ferric oxide, hydrated ferric hydroxide, hydrated polymeric ferric hydroxide, hydrated polymeric ferric oxyhydroxide, partially neutralized ferric salts and partially neutralized polymeric ferric salts.

In an aspect of the compound as described above, the iron meglumine has a weight average molecular weight distribution starting at about 400 daltons to about 7,000,000 daltons.

In an aspect, the compound as described above contains iron and meglumine in a molar ratio of iron to meglumine starting from about 1:0.1 to about 1:20 respectively.

In an aspect, the compound as described above has a formula:

(Fe)x·(O)y·(OH)z·(H2O)m·(MGM)n·(L)k

wherein “MGM” represents meglumine having molecular formula C7H17NO5
“L” represents a ligand selected from a group including carbohydrate, dextran, poly carbohydrate, sugar, dextrose, gluconic acid, derivatives of carbohydrates, substituted carbohydrates, anion, functionalized carbohydrates, starch, acid, base, amine, organic base, organic acid, organic amino compounds,
“Fe” represents predominantly Ferric iron
“O” represents oxygen
“OH” represents hydroxy group
“H2O” represents water molecule
“x”, “y”, “z” and “n” are integers having a range from about 1 to about 200,000 “k” and “m” are integers having a range of from about 0 to about 200,000

In certain embodiments, the present invention relates to a pharmaceutical composition comprising the compound as described above.

In an aspect, the pharmaceutical composition as described above is administered parenterally. In another aspect, the pharmaceutical composition as described above is administered orally.

In an aspect, the pharmaceutical composition as described above is used to treat iron deficient anemia. In another aspect, the pharmaceutical composition as described above is used as an oral phosphate binder for reducing intake of dietary phosphate in a patient in need thereof.

In an aspect, the pharmaceutical composition as described above is a solution having a concentration of iron at a level starting from about 1 mg/ml to about 300 mg/mL, and wherein said composition is administered parenterally.

In an aspect, the pharmaceutical composition as described above is administered orally, and wherein said composition is administered alone or by combining with one or more pharmaceutically acceptable inactive ingredients.

In certain aspects, the present invention relates to a process of making the compound as described above, wherein the process involves the step of combining an iron source with meglumine.

In an aspect of the process as described above, the iron source is one or more selected from a group consisting of ferric iron, ferrous iron, iron hydroxide, anionic salt of ferric iron, anionic salt of ferrous iron.

In certain embodiments, the present invention relates to a method of treating iron deficient anemia by administering iron meglumine composition as described above at a desired dosage level.

In an aspect, the method as described above comprises administering iron meglumine composition in combination with other drugs.

In an aspect, the method as described above is for reducing phosphate by orally administering the iron meglumine composition as described above at a desired dosage level. In another aspect, the method as described above is for treating iron deficient anemia by administering the pharmaceutical composition as described above.

In an aspect, the compound as described above is a combination of one or more compounds containing iron and meglumine.

In an aspect, the compound as described above is one or more of the type such as, addition compound, hydrogen bonded compound, inclusion complex, occlusion complex, coordinate bonded compound, inorganic complex, covalent bonded compound, metal-organic compound, metal-organic complex, multi-layer compound, core-shell compound.

### DESCRIPTION OF FIGURES

FIG. 1 shows a GPC molecular weight distribution Chromatogram of Ferric meglumine obtained by the process of this invention as described in Example 1

FIG. 2 shows a GPC molecular weight distribution Chromatogram of Ferric meglumine obtained by the process of this invention as described in Example 2

FIG. 3 shows a GPC molecular weight distribution Chromatogram of Ferric meglumine obtained by the process of this invention as described in Example 9

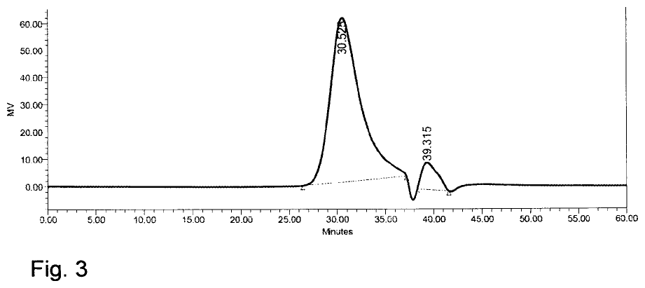

### DETAILED DESCRIPTION

Unless defined otherwise, all the technical and scientific terms used herein have the same meaning as commonly known by a person skilled in the art. In case of conflict, the definitions provided herein will prevail. Unless specified otherwise, all the percentages, portions and ratios in the present invention are on weight basis. The term “product” or “pharmaceutical product” used herein means a “pharmaceutical composition”; thus an injectable “product” as recited herein means an injectable “composition.”

### Ferric Meglumine as Parenteral Iron Supplement

An embodiment of this invention is the pharmaceutical composition comprising the compound ferric meglumine. An embodiment of the present invention involves using the composition of ferric meglumine for parenteral administration as an iron supplement.

Ferric meglumine is slightly soluble in methanol and insoluble in acetone. Ferric Meglumine is freely soluble in water to produce a dark red, dark brownish red solution. Such a solution can be aseptically processed and/or terminally sterilized to manufacture the injectable product in suitable container. Such a solution of Ferric meglumine can be a concentrate that contains an iron concentration of about 1 mg/ml to about 200 mg/mL. For parenteral applications the iron concentration is preferred at about 1 mg/mL to about 100 mg/mL. Parenteral solutions may be prepared directly by dissolving ferric meglumine in the medium to a desired concentration of iron or by diluting the concentrate to the desired concentration of iron. The medium can be predominantly containing water, such as water for injection, sterile water, saline, dextrose injection or other compatible injectable products. Ferric meglumine solution can be administered as parenteral iron supplement. Pharmaceutically acceptable inactive ingredients can be added into the parenteral formulation.

### Preparation of Ferric Meglumine

Meglumine is soluble in water. Most of the ferric salts such as ferric chloride, ferric sulfate, ferric nitrate are soluble in water. Ferric hydroxide can be prepared by neutralizing the ferric salt with one or more base selected from the group meglumine, sodium bicarbonate, sodium carbonate, sodium hydroxide. The ferric hydroxide may be a partially neutralized ferric salt with a base, fully neutralized ferric salt with a base, ferric hydroxide, ferric oxyhydroxide, polymeric ferric hydroxide or polymeric ferric oxyhydroxide. The ferric meglumine may be prepared by reacting a ferric hydroxide with meglumine at a molar ratio of iron to meglumine at least 1:0.1 to a maximum of 1:1000 The ferric hydroxide may be a partially neutralized ferric salt with a base, fully neutralized ferric salt with a base, ferric hydroxide, ferric oxyhydroxide, polymeric ferric hydroxide or polymeric ferric oxyhydroxide. The ferric meglumine may be prepared by reacting a ferric hydroxide with meglumine at a molar ratio of iron to meglumine at least 1:0.1. Meglumine can be used at a molar ratio from about 0.2 to about 20. Optionally the reaction temperature can be adjusted between 5° C. to about a refluxing temperature (about 100-105° C.) to facilitate the reaction to product ferric meglumine. Meglumine itself may act as the reactant and a base to adjust pH. Optional bases such as sodium hydroxide, sodium carbonate can be used to adjust pH of the reaction medium as a co-base. Optional acids such as hydrochloric acid, acetic acid, citric acid, or any pharmaceutically acceptable acid can also be used to adjust the pH to the desired level.

### Isolation of Ferric Meglumine

Ferric meglumine prepared as a dark red or dark brown or reddish brown solution can be mixed with an organic solvent to precipitate ferric meglumine. The precipitated ferric meglumine may be isolated by filtration, sedimentation, or centrifuging. The isolated ferric meglumine may be dried at a suitable temperature from between 5° C. to a temperature below the decomposition point of ferric meglumine. Optionally vacuum can be applied along with the set temperature to facilitate the drying process. The dried ferric meglumine can be stored in suitable container for further use.

### Purification of Ferric Meglumine

The ferric meglumine prepared as stated in this invention, may be purified to remove excess base, and to reach a suitable pH. The purification shall involve reprecipitation, recrystallisation, dialysis, and or ultra-centrifuge to remove unwanted materials and or by evaporation or solvent reprecipitation of the ferric meglumine solution or concentrate. Such purification step can be useful to remove excess meglumine. Preferably the purification process involves dissolving the crude ferric meglumine in an aqueous medium and precipitated by mixing with one or more organic solvent.

In certain aspects, the present invention provides a method for the formation of ferric meglumine. Preferably, ferric meglumine is prepared by reacting an iron species with meglumine. The reaction can be performed in a solvent medium. Optionally the iron species can be substantially free from other metallic contamination as controlled by standard limit for elemental impurities specified in US pharmacopoeia for parenteral products. Optionally the iron species can be substantially free from anions which are part of the Ferric iron source. The iron species can be ferric salt, partially neutralized ferric salt with a base, fully neutralized ferric salt with a base, ferric hydroxide, ferric oxyhydroxide, polymeric ferric hydroxide or polymeric ferric oxyhydroxide. Optionally the Iron species can be a mixture of ferric and ferrous compounds. The base used for neutralizing ferric salt is a sodium base such as sodium hydroxide, sodium bicarbonate, sodium carbonate, and or an organic amine base. The amine base can be meglumine itself.

### Preparation of a Composition of Ferric Meglumine as an Injectable Product

Ferric meglumine purified solid or solution or concentrate may be made in to aqueous solution containing a concentration of Iron between about 0.1 mg/mL to about 200 mg/mL. Such solution shall be aseptically processed filled into suitable containers such as vials, ampoules, syringes, bottles or bags suitable for parenteral compositions. Ferric meglumine solution in bulk, or as filled in suitable container closure system may be subjected to sterilization. The sterilization can be performed by either one or more acceptable techniques such as, aseptic filtration through below 0.5 micron filter or combination of filters. Optionally the sterilization can be performed by terminal dry heat/steam sterilization, gamma ray sterilization etc. In such parenteral preparation, ferric melamine can be used alone or in compilation with other drugs and or pharmaceutical inactive ingredients. The other drugs can be of similar iron based drugs or any other drug used for other therapeutic condition. Similar parenteral products can be obtained by employing Ferric glutamine or ferric glucosamine instead ferric meglumine, or combination thereof.

The compound according to this invention is

(Fe)ₓ·(O)_{y}·(OH)_{z}·(H₂O)ₘ·(MGM)ₙ·(L)ₖ

wherein “MGM” represents meglumine having molecular formula C₇H₁₇NO₅;
“L” represents a ligand selected from a group including carbohydrate, dextran, poly carbohydrate, sugar, dextrose, gluconic acid, derivatives of carbohydrates, substituted carbohydrates, anion, functionalized carbohydrates, starch, acid, base, amine, organic base, organic acid, organic amino compounds;
“Fe” represents predominantly Ferric iron;
“O” represents oxygen;
“OH” represents hydroxy group;
“H₂O” represents water molecule;
“X”, “y”, “z” and “n” are integers having a range from about 1 to about 200,000;
“k” and “m” are an integer having a range of from about 0 to about 200,000.

### Preparation of Composition of Ferric Meglumine for Oral Administration

Ferric meglumine purified solid or solution or concentrate may be made in to suitable oral dosage form such as solution, suspension, powder, tablet, capsules, or combination thereof. In such oral preparation, ferric meglumine can be used alone or in combination with other drugs and or pharmaceutical inactive ingredients. The other drugs can be of similar iron based drugs or any other drug used for other therapeutic condition. The dose size can be adjusted based on the desired iron per dose acceptable to a patient.

According to one embodiment of the invention, the average molecular weight of the solution of ferric meglumine complex is a distribution of molecular weight in the range from 400 to 7,000,000 Daltons as determined by gel permeation chromatography.

According to embodiment of the invention, solid ferric meglumine can be isolated by evaporation of the solution containing ferric meglumine or by precipitation on adding a counter solvent to a solution containing ferric meglumine. Such isolated sold ferric meglumine can be dried and stored.

Solid or solution of ferric meglumine can be used as oral iron supplement at a desired dosage level. Such orally administrable iron supplements containing ferric meglumine can be made as powder, beads, granules, chewing gum, solution, suspension, tablet, capsule, liquid filled capsule, tablet or beads filled capsule. Pharmaceutically acceptable inactive ingredients can be added into the oral formulation.

Solid or solution of ferric meglumine can be used as phosphate binders at a desired dosage level. Such orally administrable phosphate binders containing ferric meglumine can be made as powder, beads, granules, chewing gum, solution, suspension, tablet, capsule, liquid filled capsule, tablet or beads filled capsule. Pharmaceutically acceptable inactive ingredients can be added into the oral formulation.

### Example 1: Preparation of Ferric Meglumine

Part (a) Ferric chloride hexahydrate (27 g, 0.1 mol) was dissolved in deionized water (50 mL) at a temperature of about 10° C. To the ferric chloride solution, an aqueous solution of sodium carbonate 20% w/v was added with stirring, the pH raises with effervescence, and at about a pH about 3.6 the reaction mass becomes thick indicating the formation of ferric hydroxide. The suspended precipitate was adjusted to a pH of about 5 by addicting sodium carbonate solution, isolated by centrifuge. The isolated ferric hydroxide was further washed with water to remove sodium chloride. The wet ferric hydroxide is used up for further reaction with meglumine.

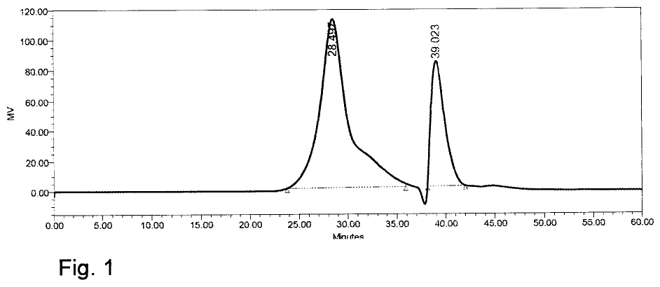

Part (b) To an aqueous suspension of ferric hydroxide obtained in Part (a) added Meglumine (5 g, 0.025 mol) and the temperature of the mixture was raised to about 80 deg C. The reaction mixture becomes clear dark red solution. The pH was about 10. The resulting solution was filtered through a series of filters 5 micron, 0.45 micron and 0.22 micron porosity and filled into vials, stoppered and sealed. The clear solution was tested by gel permeation chromatography about found to contain a wide peak having a polymeric distribution of molecular weight from about 400 daltons to about 7,000,000 daltons. The weight average molecular weight was about 225,000 daltons. (FIG. 1)

Part (c) The filled vials as obtained in part (b) was loaded into a steam sterilizer, and sterilized for a cycle of 20 min, at 121 deg C.

### Example 2: Preparation of Ferric Meglumine

Part (a) Ferric chloride hexahydrate (27 g, 0.1 mol) was dissolved in deionized water (50 mL) at a temperature of about 10° C. To the ferric chloride solution, an aqueous solution of sodium carbonate 20% w/v was added with stirring, the pH raises with effervescence, and at about a pH about 3.6 the reaction mass becomes thick indicating the formation of ferric hydroxide. The suspended precipitate was adjusted to a pH of about 5 by adding sodium carbonate solution, isolated by centrifuge. The isolated ferric hydroxide was further washed with water to remove sodium chloride. The wet ferric hydroxide is used up for further reaction with meglumine.

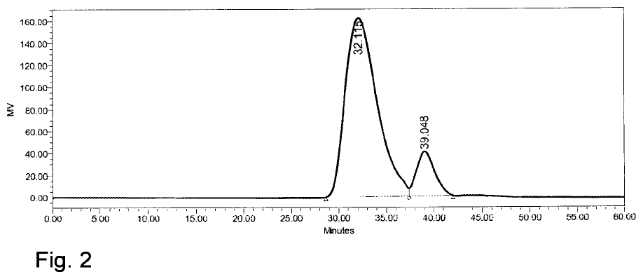

Part (b) To an aqueous suspension of ferric hydroxide obtained in Part (a) added Meglumine (31 g, 0.16 mol) and the temperature of the mixture was raised to about 60 deg C. The reaction mixture becomes clear dark red solution. The pH was about 12. The resulting solution was filtered through a series of filters 5 micron, 0.45 micron and 0.22 micron porosity and filled into vials, stoppered and sealed. The clear solution was tested by gel permeation chromatography about found to contain a wide peak having a polymeric distribution of molecular weight from about 400 daltons to about 6,000,000 daltons. The weight average molecular weight was about 74,000 daltons. (FIG. 2)

Part (c) The filled vials as obtained in part (b) was loaded into a steam sterilizer and sterilized for a cycle of 20 min, at 121 deg C.

### Example 3: Preparation of Ferric Meglumine

Part (a) Ferric chloride hexahydrate (27 g, 0.1 mol) was dissolved in deionized water (50 mL) at a temperature of about 10° C. To the ferric chloride solution, an aqueous solution of sodium carbonate 20% w/v was added with stirring, the pH raises with effervescence to a pH of about 1.5. Added Meglumine (10g, 0.051 mol) and stirred.

At about a pH about 3.8 the reaction mass becomes thick indicating the formation of ferric hydroxide. The reaction mass is used up for further reaction.

Part (b) To an aqueous suspension of ferric hydroxide and meglumine obtained in Part (a) added sodium hydroxide aqueous solution (10% w/v) to a pH of about 10 and the temperature of the mixture was raised to about 60 deg C. The reaction mixture becomes dark red solution. The pH was about 9.5. The resulting solution was concentrated by removal of water by distillation and the resulting concentrate was cooled to about 5° C. To the cold solution, added methanol until the precipitation settles down. The reaction mixture was further stirred for about 2 hrs at 5° C. and the precipitated ferric meglumine solid was isolated by filtration. The wet solid was dissolved in water to form a clear solution and tested by gel permeation chromatography about found to contain a wide peak having a polymeric distribution of molecular weight from about 500 daltons to about 7,000,000 daltons. The methanolic solution is dark and found to contain about 20% of ferric meglumine product formed, showing a molecular weight distribution from about 200 daltons to about 5,000,000 daltons appearing as multi component molecular weight distribution

Part (c) The solid crude ferric meglumine obtained in part (b) was purified by dissolving in water and reprecipitated by adding methanol. The reprecipitated purified ferric meglumine was isolated by filtration and dried under vacuum for about 6 hrs at 45° C. The dried solid was tested for Iron content and found to contain about 25% w/w.

Part (d) The purified ferric meglumine (16 g) as obtained in part (c) is dissolved in water for injection to obtain a solution of volume 20 mL. The resulting solution was filtered through a series of filters 0.45 micron and 0.22 micron porosity and filled into vials, stoppered and sealed. The filled vials were steam sterilized for a cycle of 20 min, at 121° C.

### Example 4: Preparation of Ferric Meglumine

Part (a) Ferric chloride hexahydrate (27 g, 0.1 mol) was dissolved in deionized water (50 mL) at a temperature of about 10° C. To the ferric chloride solution, an aqueous solution of sodium carbonate 20% w/v was added with stirring, the pH raises with effervescence, and at about a pH about 3.6 the reaction mass becomes thick indicating the formation of ferric hydroxide. The suspended precipitate was adjusted to a pH of about 6 by adding sodium carbonate solution, isolated by filtration. The isolated ferric hydroxide was further washed with water to remove sodium chloride. The wet ferric hydroxide is used up for further reaction with meglumine.

Part (b) To an aqueous suspension of ferric hydroxide obtained in Part (a) added Meglumine (10 g, 0.051 mol) and the temperature of the mixture was raised to about 80 deg C. The reaction mixture becomes clear dark red solution. The pH was about 11. The resulting solution was filtered through a series of filters 5 micron, 0.45 micron and 0.22 micron porosity and filled into vials, stoppered and sealed. The clear solution was tested by gel permeation chromatography about found to contain a wide peak having a polymeric distribution of molecular weight from about 400 daltons to about 7,000,000 daltons.

Part (c) The filled vials as obtained in part (b) was loaded into a steam sterilizer, and sterilized for a cycle of 20 min, at 121 deg C.

### Example 5: Preparation of Ferric Meglumine

Part (a) Ferric chloride hexahydrate (27 g, 0.1 mol) was dissolved in deionized water (50 mL) at a temperature of about 10° C. To the ferric chloride solution, an aqueous solution of sodium carbonate 20% w/v was added with stirring, the pH raises with effervescence, and the ferric chloride solution is partially neutralized to a pH of about 1.5. Added Meglumine (10 g, 0.051 mol) and stirred. At about a pH about 3.8 the reaction mass becomes thick indicating the formation of ferric hydroxide. The reaction mass is used up for further reaction.

Part (b) To an aqueous suspension of ferric hydroxide and meglumine obtained in Part (a) added sodium hydroxide aqueous solution (10% w/v) to a pH of about 10 and the temperature of the mixture was raised to about 60 deg C. The reaction mixture becomes clear dark red solution. The pH was about 9.5. The resulting solution was concentrated by removal of water by distillation and the resulting concentrate was cooled to ambient temperature about 20° C. Adjusted the pH of the mixture to about 6.5 using 5molar hydrochloric acid. The reaction mass is further cooled to about 5° C. To the cold solution, added isopropanol until the precipitation settles down. The reaction mixture was further stirred for about 2 hrs at 5° C. and the precipitated ferric meglumine solid was isolated by filtration. The wet solid was dissolved in water to form a clear solution and tested by gel permeation chromatography about found to contain a wide peak having a polymeric distribution of molecular weight from about 400 daltons to about 7,000,000 daltons.

Part (c) The solid crude ferric meglumine obtained in part (b) was purified by dissolving in water and reprecipitated by adding acetone. The reprecipitated purified ferric meglumine was isolated by filtration and dried under vacuum for about 6 hrs at 45° C. The dried solid was tested for Iron content and found to contain about 60% w/w.

Part (d) The purified ferric meglumine (10 g) as obtained in part (c) is dissolved in water for injection to obtain a solution of volume 400 mL. The resulting solution was filtered through a series of filters 0.45 micron and 0.22 micron porosity and filled into vials, stoppered and sealed.

### Example 6: Preparation of Ferric Meglumine

Part (a) Ferric chloride solution 40% w/v substantially free from elemental impurities (41 g, 0.1 mol) was diluted with deionized water (20 mL) at a temperature of about 20° C. To the ferric chloride solution, an aqueous solution of sodium carbonate 20% w/v was added with stirring, the pH raises with effervescence, and the ferric chloride solution is partially neutralized to a pH of about 1.5. Added Meglumine (10 g, 0.051 mol) and stirred. At about a pH about 4.2 the reaction mass becomes thick indicating the formation of ferric hydroxide. The reaction mass is used up for further reaction.

Part (b) To an aqueous suspension of ferric hydroxide and meglumine obtained in Part (a) added sodium hydroxide aqueous solution (10% w/v) to a pH of about 10 and the temperature of the mixture was raised to about 60 deg C. The reaction mixture becomes clear dark red solution. The resulting solution was concentrated by removal of water by distillation and the resulting concentrate was cooled to ambient temperature about 20° C. Adjusted the pH of the mixture to about 6.5 using 5molar hydrochloric acid. The reaction mass is further cooled to about 5° C. To the cold solution, added ethanol until the precipitation settles down. The reaction mixture was further stirred for about 2 hrs at 5° C. and the precipitated ferric meglumine solid was isolated by filtration. The wet solid was dissolved in water to form a clear solution and tested by gel permeation chromatography anout found to contain a wide peak having a polymeric distribution of molecular weight from about 400 daltons to about 7,000,000 daltons.

Part (c) The solid crude ferric meglumine obtained in part (b) was purified by dissolving in water and reprecipitated by adding ethanol. The reprecipitated purified ferric meglumine was isolated by filtration and dried under vacuum for about 6 hrs at 45° C. The dried solid was tested for Iron content and found to contain about 45% w/w.

Part (d) The purified ferric meglumine (5g) as obtained in part (c) is dissolved in water for injection to obtain a solution of volume 200mL. The resulting solution was filtered through a series of filters 0.45 micron and 0.22 micron porosity and filled into vials, stoppered and sealed.

### Example 7: Preparation of Ferric Meglumine

Part (a) Ferric chloride hexahydrate (27 g, 0.1 mol) was dissolved in deionized water (75 mL) at a temperature of about 20° C. To the ferric chloride solution, an aqueous solution of sodium carbonate 20% w/v was added with stirring, to a pH about 5, the reaction mass becomes thick indicating the formation of ferric hydroxide. The suspended precipitate was adjusted to a pH of about 6.5 by adding sodium carbonate solution, isolated by centrifuge. The isolated ferric hydroxide was further washed with water to remove sodium chloride. The wet ferric hydroxide is used up for further reaction with meglumine.

Part (b) To an aqueous suspension of ferric hydroxide obtained in Part (a) added Meglumine (5 g, 0.025 mol) and the temperature of the mixture was raised to about 80 deg C. The reaction mixture becomes clear dark red solution. The pH was about 10. One part of the resulting solution was filtered through a series of filters 5 micron, 0.45 micron and 0.22 micron porosity and filled into vials, stoppered and sealed. The clear solution was tested by gel permeation chromatography about found to contain a wide peak having a polymeric distribution of molecular weight from about 400 daltons to about 7,000,000 daltons. The second part was mixed with acetone to get a dark brown precipitate. The precipitated ferric meglumine was isolated and dried. The dried ferric meglumine containes about 40% of iron by weight, is dissolved in water and added additional meglumine to form a solution containing about 50 mg iron per mL. Ferric meglumine solution containing iron at about 50 mg/mL was filtered through a series of filters 5 micron, 0.45 micron and 0.22 micron porosity and filled into vials, stoppered and sealed

Part (c) The filled vials as obtained in part (b) was loaded into a steam sterilizer, and sterilised for a cycle of 20 min, at 121 deg C.

### Example 8: Preparation of Ferric Meglumine

Part (a) To an aqueous solution of Ferric chloride equivalent to (27 g, 0.1 mol) of Ferric chloride hexahydrate at a temperature of about 10° C. to 20° C. an aqueous solution of sodium carbonate 15% w/v was added with stirring, to a pH about 5, the reaction mass becomes thick indicating the formation of polymeric ferric oxyhydroxide. The suspended precipitate was adjusted to a pH of about 6.0 by adding sodium carbonate solution, isolated by centrifuge. The isolated polymeric ferric oxyhydroxide was further washed with water to remove sodium chloride. The wet polymerric ferric oxyhydroxide is used up for further reaction with meglumine.

Part (b) To an aqueous suspension of polymeric ferric oxyhydroxide obtained in Part (a) added aqueous solution containing Meglumine (5 g, 0.025 mol) and the temperature of the mixture was raised to about 80 deg C. The reaction mixture becomes clear dark red solution. The pH was about 10. The Ferric meglumine solution was concentrated to a desired concentration of Iron from about 10 mg/mL to about 200 mg/mL. One part of the resulting solution was filtered through a series of filters 5 micron, 0.45 micron and 0.22 micron porosity and filled into vials, stoppered and sealed. The clear solution was tested by gel permeation chromatography about found to contain a wide peak having a polymeric distribution of molecular weight from about 500 daltons to about 7,000,000 daltons. The second part was mixed with acetone to get a dark brown precipitate. The precipitated ferric meglumine was isolated and dried. The dried ferric meglumine contains about 45% of iron by weight, is dissolved in water and added additional meglumine (10 mg/mL) and sucrose (10 mg/mL) to form a solution containing about 50 mg Iron per mL. Ferric meglumine solution containing iron at about 50 mg/mL was filtered through a series of filters 5 micron, 0.45 micron and 0.22 micron porosity and filled into vials, stoppered and sealed

Part (c) The filled vials as obtained in part (b) was loaded into a steam sterilizer and sterilised for a cycle of 20 min, at 121 deg C.

### Example 9: Preparation of Ferric Meglumine Injection

Part (a) To an aqueous solution of Ferric chloride equivalent to (270 g, 1 mol) of Ferric chloride hexahydrate at a temperature of about 10° C. to 20° C. an aqueous solution of sodium carbonate 15% w/v was added with stirring, to a pH about 5, the reaction mass becomes thick indicating the formation of polymeric ferric oxyhydroxide. The suspended precipitate was adjusted to a pH of about 6.0 by adding sodium carbonate solution, isolated by centrifuge. The isolated polymeric ferric oxyhydroxide was further washed with water to remove sodium chloride. The wet polymerric ferric oxyhydroxide is used up for further reaction with meglumine.

Part (b) To an aqueous suspension of polymeric ferric oxyhydroxide obtained in Part (a) added aqueous solution containing Meglumine (100 g, 0.51 mol) and the temperature of the mixture was raised to about 80 deg C. The reaction mixture becomes clear dark red solution. The pH was about 11. The Ferric meglumine solution was concentrated to a desired concentration of Iron from about 10 mg/mL to about 200 mg/mL. The resulting solution was filtered to remove any insoluble matter.

Part (c) The resulting solution as obtained in Part (c) was diluted to a desired injectable concentration, filtered through a series of filters, 0.45 micron and 0.22 micron porosity and filled into vials, stoppered, sealed and terminally sterilised by steam at 121° C. The clear solution was tested by gel permeation chromatography about found to contain a wide peak having a polymeric distribution of molecular weight from about 500 daltons to about 7,000,000 daltons. Weight average molecular weight is about 115,000 daltons. (FIG. 2)

Part (d) An alternate injectable formulation of Ferric meglumine was made by mixing the resulting solution obtained in Part (b) with additional quantity of meglumine (2 mg/mL) and mannitol (5 mg/mL), filtered through a series of filters micron, 0.45 micron and 0.22 micron porosity and filled into vials, stoppered and sealed. The filled vials were loaded into a steam sterilizer and sterilised for a cycle of 20 min, at 121 deg C.

### Example 10: Preparation of Ferric Meglumine

Part (a) To an aqueous solution of Ferric chloride equivalent to (270 g, 1 mol) of Ferric chloride hexahydrate at a temperature of about 10° C. to 20° C. an aqueous solution of sodium carbonate 15% w/v was added with stirring, to a pH about 5, the reaction mass becomes thick indicating the formation of polymeric ferric oxyhydroxide. The suspended precipitate was adjusted to a pH of about 6.0 by adding sodium carbonate solution, isolated by centrifuge. The isolated polymeric ferric oxyhydroxide was further washed with water to remove sodium chloride. The wet polymerric ferric oxyhydroxide is used up for further reaction with meglumine.

Part (b) To an aqueous suspension of polymeric ferric oxyhydroxide obtained in Part (a) added aqueous solution containing Meglumine (50 g, 0.025 mol) and the temperature of the mixture was raised to about 80 deg C. The reaction mixture becomes clear dark red solution. The pH was about 11. The Ferric meglumine solution was concentrated to a desired concentration of Iron from about 10 mg/mL to about 200 mg/mL. The resulting solution was filtered to remove any insoluble matter. The filtered solution was mixed with acetone to get a dark brown precipitate. The precipitated ferric meglumine was isolated and dried. The dried ferric meglumine contains about 38% of iron by weight, The dried solid was purified further by dissolving in water and precipitated by adding acetone-alcohol mixture. The precipitated solid was isolated and dried. The purified ferric meglumine contains about 48% Iron by weight.

### Example 11: Preparation of Ferric Meglumine for Oral Administration

Part (a) To an aqueous solution of Ferric chloride equivalent to (270 g, 1 mol) of Ferric chloride hexahydrate at a temperature of about 10° C. to 20° C. an aqueous solution of sodium carbonate 15% w/v was added with stirring, to a pH about 5, the reaction mass becomes thick indicating the formation of polymeric ferric oxyhydroxide. The suspended precipitate was adjusted to a pH of about 6.0 by adding sodium carbonate solution, isolated by centrifuge. The isolated polymeric ferric oxyhydroxide was further washed with water to remove sodium chloride. The wet polymeric ferric oxyhydroxide is used up for further reaction with meglumine.

Part (b) To an aqueous suspension of polymeric ferric oxyhydroxide obtained in Part (a) added aqueous solution containing Meglumine (50 g, 0.025 mol) and the temperature of the mixture was raised to about 80 deg C. The reaction mixture becomes clear dark red solution. The pH was about 11. The Ferric meglumine solution was concentrated to a desired concentration of Iron from about 10 mg/mL to about 200 mg/mL. The resulting solution was filtered to remove any insoluble matter. To the filtered solution, added 50 G Sucrose and 50 g Maize starch, and stirred to mix. The mixture was added to a mixture of acetone and isopropyl alcohol. The coprecipitated mixture of ferric meglumine, sucrose and starch was isolated by centrifuge, and dried. The dried ferric meglumine coprecipitate contains about 20% of iron by weight,

Part (c) The dried solid obtained in part (b) was mixed with magnesium stearate and punched into tablet suitable for oral administration

Part (d) Alternatively the dried solid obtained in part (b) was mixed with magnesium stearate and filled into capsules suitable for oral administration

### Example 12: Preparation of Ferric Meglumine Injection

Part (a) To an aqueous solution of Ferric chloride equivalent to (27 g, 0.1 mol) of Ferric chloride hexahydrate at a temperature of about 10° C. to 20° C. an aqueous solution of sodium carbonate 15% w/v was added with stirring, to a pH about 5, the reaction mass becomes thick indicating the formation of polymeric ferric oxyhydroxide. The suspended precipitate was adjusted to a pH of about 6.0 by adding sodium carbonate solution, isolated by centrifuge. The isolated polymeric ferric oxyhydroxide was further washed with water to remove sodium chloride. The wet polymeric ferric oxyhydroxide is used up for further reaction with meglumine.

Part (b) To an aqueous suspension of polymeric ferric oxyhydroxide obtained in Part (a) added aqueous solution containing Meglumine (5 g, 0.025 mol) and the temperature of the mixture was raised to about 80 deg C. The reaction mixture becomes clear dark red solution. The pH was about 10. The Ferric meglumine solution was concentrated to a desired concentration of Iron from about 10 mg/mL to about 200 mg/mL. The pH of the solution was adjusted with hydrochloric acid to about 7.0. The resulting solution was filtered to remove any insoluble matter.

Part (c) The resulting solution as obtained in Part (c) was diluted to a desired injectable concentration, filtered through a series of filters, 0.45 micron and 0.22 micron porosity and filled into vials, stoppered, sealed and terminally sterilized by steam at 121° C. The clear solution was tested by gel permeation chromatography about found to contain a wide peak having a polymeric distribution of molecular weight from about 500 daltons to about 7,000,000 daltons.

Part (d) An alternate injectable formulation of Ferric meglumine was made by mixing the resulting solution obtained in Part (b) with and sucrose (5 mg/mL), filtered through a series of filters micron, 0.45 micron and 0.22 micron porosity and filled into vials, stoppered and sealed. The filled vials were loaded into a steam sterilizer and sterilised for a cycle of 20 min, at 121 deg C.

### Example 12: Gel Permeation Chromatography

Ferric meglumine prepared by the processes disclosed in this invention was tested for the molecular weight distribution by gel permeation chromatography. The mobile phase contains sodium phosphate monobasic 6 g and sodium phosphate dibasic 5 g dissolved in 2 Lit water and filtered. Two GPC columns (SEPAX SRT SEC 1000, 7.8 mm×300 mm and SEPAX SRT SEC 100, 7.8 mm×300 mm) were connected in series. Using suitable HPLC system with Refractive index detector the GPC chromatograms were recorded at a flow rate 0.5 mL/min and the molecular weight distribution was determined using calibration curve generated by dextran standards.

The description of the invention is broadly exemplary. Any hypothetical, ideological, practical, investigational improvements and deviations on the examples, deviations or modifications on the core concept of the invention, an extension of exemplary ideas conceived from this invention are considered to be within the scope of the invention.

1. A compound comprising iron and meglumine.

2. The compound of claim 1, wherein the compound consists of one or more components selected from a group consisting of oxide, hydroxide, water, sodium, chloride, solvent, organic impurities, and elemental impurities.

3. The compound of claim 1, wherein the compound is substantially free from organic impurities.

4. The compound of claim 1, wherein the compound is substantially free from elemental impurities or having elemental impurities at a level below the acceptance level for parenteral products.

5. A pharmaceutical composition comprising the compound of claim 1.

6. The composition of claim 5, wherein the composition is suitable for parenteral administration.

7. The composition of claim 5, wherein the composition is suitable for oral administration.

8. A method of treating iron deficient anemia, wherein the method comprises administering the composition of claim 5 to a patient in need thereof.

9. A method of reducing dietary intake of dietary phosphate, wherein the method comprises administering the composition of claim 5 as an oral phosphate binder.

10. The pharmaceutical composition of claim 5, wherein the pharmaceutical composition is a solution having a concentration of iron at a level starting from about 1 mg/mL to about 300 mg/mL, and wherein said composition is administered parenterally.

11. The pharmaceutical composition of claim 5, wherein the pharmaceutical composition is administered orally, and wherein said composition is administered alone or by combining with one or more pharmaceutically acceptable inactive ingredients.

12. The compound of claim 1, wherein said compound contains iron and meglumine in a molar ratio of iron to meglumine starting from about 1:0.1 to about 1:20 respectively.

13. A process of making the compound of claim 1, wherein the process involves the step of combining an iron source with meglumine.

14. A process of claim 13, wherein the iron source is one or more selected from a group consisting of ferric iron, ferrous iron, iron hydroxide, anionic salt of ferric iron, and anionic salt of ferrous iron.

15. The compound of claim 1, wherein the iron is ferric iron.

16. The compound of claim 1, wherein the iron is a mixture of ferric and ferrous iron.

17. The compound of claim 1, wherein the iron is present in the form of iron hydroxide.

18. The compound of claim 17, wherein the iron hydroxide is a ferric iron compound and/or combination ferric iron and ferrous iron thereof.

19. The process of claim 13, wherein the iron is anion salt of ferric iron or a mixture of anion salt of ferrous and ferric iron.

20. The process of claim 19, wherein the anion is one or more selected from the group consisting of chloride, bromide, iodide, acetate, citrate, nitrate, sulfate, and mesylate.

21. The compound of claim 1, wherein the iron is one or more selected from the group consisting of ferric hydroxide, polymeric ferric hydroxide, ferric oxyhydroxide, polymeric ferric oxyhydroxide, ferric hydroxide gel, hydrated ferric oxide, hydrated ferric hydroxide, hydrated polymeric ferric hydroxide, hydrated polymeric ferric oxyhydroxide, partially neutralized ferric salts and partially neutralized polymeric ferric salts.

22. The compound of claim 1, wherein the iron meglumine has a weight average molecular weight distribution starting at about 400 daltons to about 7,000,000 daltons.

23. A method of treating iron deficient anemia, said method comprising administering the composition of claim 5 at a desired dosage level.

24. The method of claim 23, wherein the method comprises administering iron meglumine composition in combination with other drugs.

25. A method of reducing phosphate, said method comprising orally administering the iron meglumine composition of claim 5 at a desired dosage level.

26. A method of treating iron deficient anemia, said method comprising administering a pharmaceutical composition comprising the compound of claim 1.

27. A method of reducing phosphate, said method comprising orally administering a composition comprising the compound of claim 1.

28. The compound of claim 1, wherein the compound is a combination of one or more compounds containing iron and meglumine.

29. The compound of claim 1, wherein the iron meglumine exists as one or more of the type such as, addition compound, hydrogen bonded compound, inclusion complex, occlusion complex, coordinate bonded compound, inorganic complex, covalent bonded compound, metal-organic compound, metal-organic complex, multi-layer compound, core-shell compound.

30. A compound comprising iron and meglumine having the following formula:
(Fe)ₓ·(O)_{y}19 (OH)_{z}·(H₂O)ₘ·(MGM)ₙ·(L)ₖ
wherein “MGM” represents meglumine having molecular formula C₇H₁₇NO₅;
“L” represents a ligand selected from a group including carbohydrate, dextran, poly carbohydrate, sugar, dextrose, gluconic acid, derivatives of carbohydrates, substituted carbohydrates, anion, functionalized carbohydrates, starch, acid, base, amine, organic base, organic acid, organic amino compounds;
“Fe” represents predominantly Ferric iron;
“O” represents oxygen;
“OH” represents hydroxy group;
“H2O” represents water molecule;
“x”, “y”, “z” and “n” are integers having a range from about 1 to about 200,000;
“k” and “m” are integers having a range of from about 0 to about 200,000.
